(19) United States
(12) Patent Application Publication
(10) Pub. No.: US 2009/0092690 A1
(43) Pub. Date: Apr. 9, 2009

(54) Process of making theaflavins enriched tea extract

(76) Inventors: Hsiu Wei Yang, Erie, PA (US); Shiming Li, Edison, NJ (US); Bu-xiang Sun, Guilin (CN); Ling-zhi Tang, Guilin (CN); Bo-feng Wu, Guilin (CN); Zhi-hui Wang, Guilin (CN); Zhen-peng Chen, Guilin (CN)

Correspondence Address: COOPER & DUNHAM, LLP, 30 Rockefeller Plaza, 20th Floor, NEW YORK, NY, 10112, US

(21) Appl. No.: 12/220,620

(22) Filed: Jul. 24, 2008

Related U.S. Application Data
(60) Provisional application No. 61/070,313, filed on Mar. 21, 2008.

(30) Foreign Application Priority Data
Jul. 26, 2007 (CN) 200710130799.6

(51) Int. Cl. A61K 36/82; A23F 3/34; A23L 1/29; A23F 3/16; A61P 39/06; C09K 15/08; A61K 31/352

(52) U.S. Cl. 424/729; 426/655; 426/648; 426/429; 426/546; 514/456

(57) ABSTRACT

This invention provides various products and processes such as a process of making a theaflavins enriched extract of tea having a low content of high molecular weight thearubigins which comprises extracting theaflavins from tea using ethanol to produce an extract having a high theaflavins content and a low content of high molecular weight thearubigins.

### BACKGROUND OF THE INVENTION

Catechins, theaflavins and thearubigins are polyphenolic compounds and major components of black tea and oolong tea. Theaflavins and thearubigins are tea-color materials. The approximate mean percentages of these compounds found in black tea beverages are shown in table 1:

TABLE 1
Major components of black tea beverages

| | |
|---|---|
| Catechins | 3-10 |
| Theaflavins | 3-6 |
| Thearubigins | 12-18 |

Components measured in wt % of extract solids.

Theaflavins are a class of benzo[7]annulenone compounds which are formed from oxidation reactions of polyphenolic compounds. There are 12 components in theaflavins, including theaflavin (TF), theaflavin-3-gallate (TFMG), theaflavin-3,3′-digallate (TFdiG) and theaflavin-3′-gallate (TFM′G), which are depicted by the following formula:

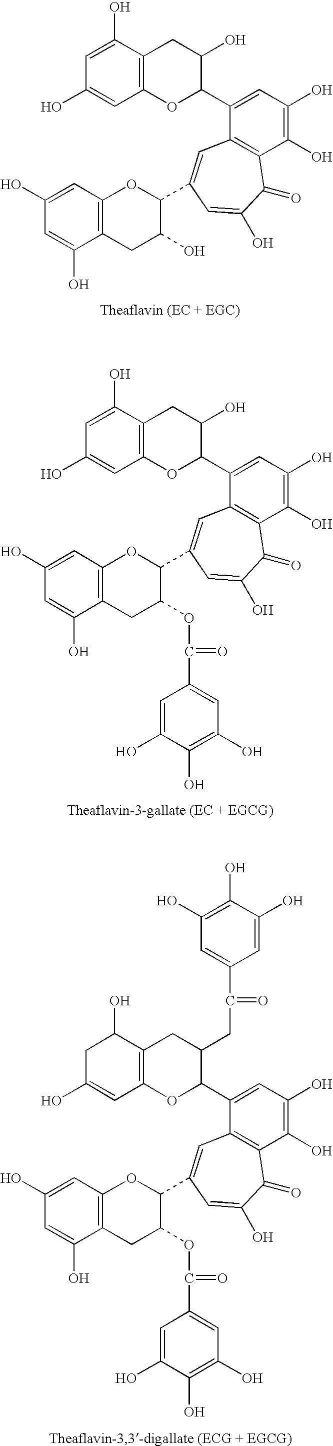

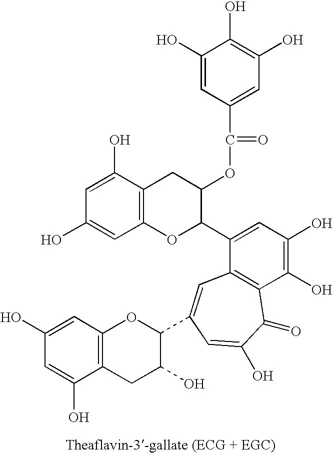

which are the four major components. Pure theaflavins are orange colors, form needle crystals, have melting points of 237-240° C., are soluble in water, methanol, ethanol, acetone, n-butanol and ethyl acetate, are slightly soluble in ethyl ether, and are insoluble in chloroform and benzene. Theaflavins in solution are clear orange in color and are slightly acidic with a pH value of about 5.7. The solution color is not affected by the pH of the tea extraction solution, but theaflavins are auto-oxidative in basic solution. The oxidation process increases with the pH value.

Thearubigins are a class of complex, inhomogeneous brown colored phenolic compounds, with a range of molecular weight of 1,000-40×10³. Due to inhomogeneity, unclear structure, and unknown properties, it is difficult to isolate and purify the thearubigins.

Tea polyphenols, including catechins and theaflavins, are known for reducing triglyceride, removing free radicals, having anti-oxidant, anti-bacteria, anti-virus, anti-tumor, anti-mutagenic, and odor removal properties, and treating cardiovascular diseases, etc. They are applied in pharmaceutical, nutraceutical and food additive fields.

Currently, the extraction of theaflavins mainly starts with black tea, tea leaves or tea polyphenols. The fermentation methods include use of chemical oxidation and polyphenol oxidase. Chemical oxidation is the oxidation of polyphenols by chemical reagents under controlled conditions. The content of theaflavins is usually high, but the chemical reagents cannot be recovered. The polyphenol oxidase method is the oxidation reaction catalyzed by polyphenol peroxidase. This method has high cost, the content of theaflavins is low, and the content of thearubigins is high. Ethyl acetate is the solvent used to extract the polyphenolic compounds, including theaflavins and thearubigins, from the initial mixture solution.

Thus there is a need to provide a method of making a high quality theaflavins enriched tea extract with low content of thearubigins from fresh green tea leaves or green tea extracts.

### SUMMARY OF THE INVENTION

This invention provides a composition comprising at least 10% by weight of total theaflavins.

This invention also provides a method of making a theaflavins enriched extract of tea having a low content of high molecular weight thearubigins which comprises extracting theaflavins from tea using ethanol to produce an extract having a high theaflavins content and a low content of high molecular weight thearubigins.

### DETAILED DESCRIPTION OF THE INVENTION

For convenience, before further description of the present invention, certain terms employed in the specification, examples and appended claims are defined here.

The term “percent by weight” of a theaflavin or theaflavins means the weight of such theaflavin or theaflavins as measured by high-performance liquid chromatography (HPLC), sometimes referred to as high-pressure liquid chromatography. While it is also possible to measure the percent by weight of total theaflavins by using UV absorption techniques, such techniques such as spectrophotometry detect ancillary materials and therefore report a higher and inaccurate percentage by weight of theaflavins than the HPLC method of measurement. Therefore, to provide the most accurate disclosure, all measurements and reporting of percentages by weight are done using HPLC method and pure substance standard for theaflavins.

The term “free of ethyl acetate” means that there is no trace of ethyl acetate. In contrast a composition that comprises an ethyl acetate extract, would contain traces of ethyl acetate and therefore is not “free of ethyl acetate” as used herein.

The term “food” includes all edible compositions regardless of form and thus includes gels, gel packs, liquids, syrups, and/or solids.

A “carrier” or “diluent” can be used to administer the compound to an animal, and can be effected or performed using any of the various methods and delivery systems known to those skilled in the art. The following non-limiting examples are provided to further illustrate the claimed invention. Compositions comprising the present invention can be formulated for administration as a food supplement using one or more fillers. Alternatively, compositions according to the present invention can be administered as conventional pharmaceuticals using one or more physiologically acceptable carriers or excipients. Compositions can be formulated for administration by any route including, but not limited to, inhalation or insufflation (through mouth or nose), oral, buccal, parenteral, vaginal, or rectal administration. For oral administration, the compositions may be added directly to foods and ingested as part of a normal meal. Compositions according to the present invention can also be administered in the form of tablets or capsules prepared by conventional means with pharmaceutically acceptable excipients such as binding agents, fillers, lubricants, disintegrants, or wetting agents. Examples of specific compounds for use in formulating tablets and capsules are described in detail in the U.S. Pharmacopeia. Tablets comprising the composition can also be coated by methods well known in the art. Liquid preparations for oral administration can also be used. Liquid preparations can be in the form of solutions, syrups, or suspensions, or a dry product for reconstitution with water or another suitable vehicle before use. Such liquid preparations can be prepared by conventional means with pharmaceutically acceptable additives such as suspending agents, emulsifying agents, non-aqueous vehicles, and preservatives. Again, specific additives are well known to those of skill and are listed in places such as the U.S. Pharmacopeia. An oral preparation may be formulated to provide controlled time release of the active nutraceutical components. For buccal administration, the extract can be formulated as a tablet or lozenge. For administration by inhalation, compositions for use in the present invention can be delivered in the form of an aerosol spray in a pressurized package or as a nebulizer, with use of suitable propellants. In the case of a pressurized aerosol, the dosage unit can be determined by providing a valve to deliver a metered dose. Parenterally administered compositions are formulated to allow for injection, either as a bolus or as a continuous infusion. Formulations for injection can be prepared in unit dosage forms, such as ampoules, or in multi-dose units, with added preservatives. The compositions for injection can be in the form of suspensions, solutions, or emulsions, in either oily or aqueous vehicles. They may also contain formulatory agents such as suspending agents, stabilizing agents, and/or dispersing agents. The active ingredient may also be presented in powder form for reconstitution with a suitable vehicle before use. Specific examples of formulating agents for parenteral injection are found in the U.S. Pharmacopeia. For rectal administration or vaginal administration, compositions for use in one of the present invention can be formulated as suppositories, creams, gels, or retention enemas. As one skilled in the art would recognize, suppositories may avoid first pass metabolism.

For dietary supplements, the extract can be added in concentrations up to 5% by weight and mixed according to methods routine in the art. Dietary supplements for animals can be prepared in a variety of forms including, but not limited to, liquid, powder, or solid pill forms.

Thus the invention provides a composition that may comprise at least 10% by weight of total theaflavins.

This invention provides a composition that may comprise a pharmaceutically acceptable carrier or diluent.

This invention provides a carrier that may be a food product. This invention also provides for a carrier that may be a beverage, a dietary supplement, a capsule, a tablet, a lozenge, a coated tablet, a solution, a syrup, or a suspension.

This invention provides for a composition that is a nutraceutical.

This invention provides a composition that may be a black tea extract. This invention also provides for a composition that may be an extract of oolong tea or green tea.

This invention provides a composition that may be an ethanol extract of tea.

This invention provides for a composition that is free of ethyl acetate.

This invention provides for a composition that contains at least 60% by weight of total theaflavins.

This invention provides a composition that may be a nutraceutical.

This invention provides a composition that may comprise at least either 1% theaflavin, 5% theaflavin-3-gallate, 1% theaflavin-3′-gallate and/or 3% theaflavin-3,3′-digallate by weight.

This invention provides a composition that may comprise from about 12% to about 17% by weight theaflavin-3-gallate, from about 3% to about 8% theaflavin-3′-gallate and/or from about 9% to about 14% by weight theaflavin-3,3′-digallate.

This invention provides for a composition that may comprise about 440 mg of tea extract.

This invention provides for a composition that may comprise about 175 mg of total theaflavins.

This invention provides for a composition that may comprise not more than 0.5% by weight caffeine.

This invention also provides a method of making a theaflavins enriched extract of tea having a low content of high molecular weight thearubigins which comprises extracting theaflavins from tea using ethanol to produce an extract having a high theaflavins content and a low content of high molecular weight thearubigins.

This invention provides for a method of making a theaflavins enriched extract of tea wherein the tea is fermented in a tank containing water and fruit or vegetable juice.

This invention provides for a method of making a theaflavins enriched extract of tea wherein tea is fermented and mixed with ethanol, insoluble materials are removed and the remaining solution is isolated.

This invention provides for a method of making a theaflavins enriched extract of tea wherein tea is fermented and mixed with ethanol, insoluble materials are removed and the remaining solution is isolated and concentrated to remove ethanol, passed through an ion exchange column to remove caffeine, and the water eluate which comprises a decaffeinated theaflavin solution is collected.

This invention provides for a method of making a theaflavins enriched extract of tea wherein tea is fermented and mixed with ethanol, insoluble materials are removed and the remaining solution is isolated and concentrated to remove ethanol, passed through an ion exchange column to remove caffeine, and the water eluent which comprises a decaffeinated theaflavin solution is collected and applied to an adsorption resin which may then be washed with a 25-35% ethanol solution to remove high molecular weight thearubigins, after which theaflavins and low molecular weight thearubigins may be eluted with a 45-55% ethanol solution.

This invention provides for a method of making a theaflavins enriched extract of tea wherein the extract may contain as high as 70% theaflavins and above 30% low molecular weight thearubigins.

This invention provides for a method of making a theaflavins enriched extract of tea wherein the extract may contain less than 0.5% caffeine.

This invention provides for a method of making a theaflavins enriched extract of tea wherein the extract may be made from fresh green tea.

This invention provides for a method of making a theaflavins enriched extract of tea wherein the extract may be made from a green tea extract.

This invention provides for a method of making a theaflavins enriched extract of tea wherein the tea is fermented in a tank containing water and fruit or vegetable juice wherein the fruit juices or vegetable juices are fresh loquat juice, fresh pear juice, fresh blueberry juice, fresh apple juice, fresh grape juice, fresh plum juice or fresh eggplant juice that were obtained from fresh fruit or vegetables.

This invention provides for a method of making a theaflavins enriched extract of tea wherein tea is fermented and mixed with ethanol, insoluble materials are removed, the solution is isolated and concentrated to remove ethanol, and is then passed through an ion exchange column to remove caffeine wherein the ion exchange column may be a strong acid cation resin or a weak acid cation resin.

This invention provides for a method of making a theaflavins enriched extract of tea wherein tea is fermented and mixed with ethanol, insoluble materials are removed and the remaining solution is isolated and concentrated to remove ethanol, passed through an ion exchange column to remove caffeine, and the water eluate which comprises a decaffeinated theaflavin solution is collected and applied to an adsorption resin which may then be washed with a 25-35% ethanol solution to remove high molecular weight thearubigins, after which theaflavins and low molecular weight thearubigins may be eluted with a 45-55% ethanol solution, wherein the adsorption resin may be washed with 2-5 times the amount of water to the volume of resin.

This invention provides for a method of making a theaflavins enriched extract of tea wherein tea is fermented and mixed with ethanol, insoluble materials are removed and the remaining solution is isolated and concentrated to remove ethanol, passed through an ion exchange column to remove caffeine, and the water eluate which comprises a decaffeinated theaflavin solution is collected and applied to an adsorption resin which may then be washed with a 25-35% ethanol solution to remove high molecular weight thearubigins, after which theaflavins and low molecular weight thearubigins may be eluted with a 45-55% ethanol solution, wherein the adsorption resin may be a polystyrene large hole adsorption resin.

This invention provides for a method of making a theaflavins enriched extract of tea wherein tea is fermented and mixed with ethanol, insoluble materials are removed and the remaining solution is isolated and concentrated to remove ethanol, passed through an ion exchange column to remove caffeine, and the water eluate which comprises a decaffeinated theaflavin solution is collected and applied to an adsorption resin which may then be washed with a 25-35% ethanol solution to remove high molecular weight thearubigins, after which theaflavins and low molecular weight thearubigins may be eluted with a 45-55% ethanol solution, wherein the theaflavin eluate may be concentrated, spray-dried and ground.

The following Examples are set forth to aid in an understanding of the subject matter of this disclosure, but are not intended to, and should not be construed to, limit in any way the claims which follow thereafter.

### EXAMPLE 1

### Synopsis

This invention provides a novel method of making theaflavins enriched tea extract starting from fresh leaves of Camellia sinensis or green tea extracts. This novel method includes separation steps that reduce considerably the amount of thearubigins with high molecular weight. Thearubigins are the predominant compounds found in black tea with molecular weights ranging from 1,000 to 40,000. Thearubigins of high molecular weight are not as biologically active as theaflavins. Also, the color of theaflavin enriched tea extracts with a lower content of high molecular weight thearubigins is more suitable for application in food and beverages. The color of pure theaflavins is orange while the color of thearubigins ranges from brown to dark-brown. The separation steps also allow the use of ethyl alcohol as the organic solvent rather than ethyl acetate. Conventional methods of making theaflavins use ethyl acetate as the organic solvent. Because ethyl alcohol exhibits the lowest toxicity in long-term animal studies, it is the preferred organic solvent for the preparation of extracts for human consumption. Furthermore, these separation steps can reduce the caffeine content to less than 0.5%. The end product is a low cost high yield theaflavins enriched tea extract with low amounts of high molecular weight thearubigins, consistent desirable color, and caffeine content as low as 0.5%.

The present invention discloses a method of making a low cost high yield theaflavins enriched tea extract with theaflavins content as high as 70%, consistent desirable color, and caffeine content as low as 0.5%.

### Materials and Methods

This method includes the following steps:

### Treatment of the Starting Materials: Fresh Green Tea Leaves

1) Foreign matter and other impurities were removed from fresh tea leaves. Then, the fresh green tea leaves were washed and withered in a dry and weak light place for 8-12 h.
   2) The withered green tea leaves were crushed.

### Tea Fermentation Process—Formation of Theaflavins and Thearubigins

1) The crushed leaves were put in a fermentation tank. Water was added in the tank in the amount of 3-8 times the volume of crushed tea leaves. Fruit or vegetable juice was added in the tank in the amount of 0.5-3 times the volume of crushed green tea leaves. At temperature 35-45° C., pH=4.5-5.5, air was introduced. This natural fermentation process was maintained for 20-40 min.
2) At the end of fermentation, the air flow was stopped and the temperature was raised to 100° C. rapidly and kept for 15-30 min. A solution of 95% ethanol was added to the tank and the entire mixture was stirred for 20-40 minutes. The ratio of 95% ethanol to the initial mixture was between 1:1 and 3:1. Then, the mixture was centrifuged to separate the supernatant clear liquid and precipitates.
3) The precipitates were washed again in a tank with a solution of 50% ethanol for 30 min. The ratio of 50% ethanol to precipitates mixture was between 2:1 and 3:1. This mixture was centrifuged to separate supernatant clear liquid from precipitates.
4) The supernatant liquids of step 2 and 3 were combined and then, concentrated to remove ethanol. The concentrate was centrifuged to separate supernatant clear liquid from precipitates. The precipitates were dried to yield a material containing more than 30% thearubigins.

### Decaffeination

The supernatant clear liquid obtained from step 4 was passed through an ion exchange column. The water eluate was collected to give the decaffeinated theaflavin solution.

### Purification of Theaflavins and Thearubigins

The decaffeinated theaflavin solution was applied onto an adsorption resin. The resin was washed with water. The amount of water used was 2-5 times to the volume of resin. The tea compounds that were not adsorbed with resin were removed. Thearubigins of high molecular weight were eluted with 25-35% ethanol, which was the first elution liquid. Then, theaflavins and thearubigins of low molecular weight were eluted with 45-55% ethanol, which was the second elution liquid. The elution liquids were concentrated and alcohol recovered. The concentrated solutions were spray-dried and ground to give desired products.

From the first elution liquid, the content of high molecular weight thearubigins was above 70%. From the second elution liquid, the content of theaflavins was above 60% and low molecular weight thearubigins was above 30%.

This invention states that the starting material is either fresh plucked green tea leaves or green tea extracts.

According to this invention, the fruit juices or vegetable juices that may be used include but are not limited to fresh loquat juice, fresh pear juice, fresh blueberry juice, fresh apple juice, fresh grape juice, fresh plum juice or fresh eggplant juice that were obtained from fresh fruit or vegetables.

This invention states that the ion exchange resin column is a strong acid cation resin or weak acid cation resin.

This invention states that the stated adsorption resin is a polystyrene resin.

This invention provides a low cost high yield method for making theaflavins enriched tea extract with low content of high molecular weight thearubigins from fresh green tea leaves or green tea extracts. This process also avoids the use of ethyl acetate solvent. Contents of theaflavins and thearubigins with different molecular weights can be obtained through controlling the process conditions. The yield and the content of theaflavins are high.

### APPLICATION EXAMPLES AND RESULTS

### Example 1

### Treatment of the Starting Materials: Fresh Green Tea Leaves

1. Foreign matter and other impurities were removed from fresh tea leaves. Then, the fresh green tea leaves were washed and withered in a dry and weak light place for 8 h.
2. The withered green tea leaves were crushed.

### Tea Fermentation Process—Formation of Theaflavins and Thearubigins

1. The crushed leaves were put in a fermentation tank. Water was added in the tank in the amount of 3 times the volume of crushed tea leaves. Loquat juice was added in the tank in the amount of 0.5 times the volume of crushed green tea leaves. At temperature 35-45° C., pH=4.5, air was introduced. This natural fermentation process was maintained for 20 min.
2. At the end of fermentation, the air flow was stopped and the temperature was raised to 100° C. rapidly and kept for 15 min. A solution of 95% ethanol was added to the tank and the entire mixture was stirred for 20 minutes. The ratio of 95% ethanol to the initial mixture was 1:1. Then, the mixture was centrifuged to separate the supernatant clear liquid and precipitates.
3. The precipitates were washed again in a tank with a solution of 50% ethanol for 30 min. The ratio of 50% ethanol to precipitates mixture was 2:1. This mixture was centrifuged to separate supernatant clear liquid from precipitates.
4. The supernatant liquids of step 2 and 3 were combined and then, concentrated to remove ethanol. The concentrate was centrifuged to separate supernatant clear liquid from precipitates. The precipitates were dried to yield a material containing more than 30% thearubigins.

### Decaffeination

The supernatant clear liquid obtained from step 4 was passed through a strong acid ion exchange column. The water eluate was collected to give the decaffeinated theaflavin solution.

### Purification of Theaflavins and Thearubigins

The decaffeinated theaflavin solution was applied onto an adsorption resin (Model 101, large hole adsorption resin. Manufacturer: Xian Lanxiao Scientific Ltd, Xian, China). The resin was washed with water. The amount of water used was 2 times to the volume of resin. The tea compounds that were not assorted with resin were removed. Thearubigins of high molecular weight were eluted with 25% ethanol, which was the first elution liquid (1). Then, theaflavins and thearubigins of low molecular weight were eluted with 45% ethanol, which was the second elution liquid (2). The elution liquids were concentrated and alcohol recovered. The concentrated solutions were spray-dried and ground to give desired products.

From the first elution liquid (1), the content of high molecular weight thearubigins was above 70%. From the second elution liquid, the content of theaflavins was above 60% and low molecular weight thearubigins was above 30%.

### Example 2

### Treatment of the Starting Materials: Fresh Green Tea Leaves

1. Foreign matter and other impurities were removed from fresh tea leaves. Then, the fresh green tea leaves were washed and withered in a dry, low-light environment for 10 h.
2. The withered green tea leaves were crushed.

### Tea Fermentation Process—Formation of Theaflavins and Thearubigins

1. The crushed leaves were put in a fermentation tank. Water was added in the tank in the amount of 5 times the volume of crushed tea leaves. Apple juice was added in the tank in the amount of 1.0 times the volume of crushed green tea leaves. At temperature 40° C., pH=5.0, air was introduced. This natural fermentation process was maintained for 30 min.
2. At the end of fermentation, the air flow was stopped and the temperature was raised to 100° C. rapidly and kept for 20 min. A solution of 95% ethanol was added to the tank and the entire mixture was stirred for 30 minutes. The ratio of 95% ethanol to the initial mixture was 2:1. Then, the mixture was centrifuged to separate the supernatant clear liquid and precipitates.
3. The precipitates were washed again in a tank with a solution of 50% ethanol for 30 min. The ratio of 50% ethanol to precipitates mixture was 2:1. This mixture was centrifuged to separate supernatant clear liquid from precipitates.
4. The supernatant liquids of step 2 and 3 were combined and then, concentrated to remove ethanol. The concentrate was centrifuged to separate supernatant clear liquid from precipitates. The precipitates were dried to yield a material containing more than 30% thearubigins.

### Decaffeination

The supernatant clear liquid obtained from step 4 was passed through a strong acid ion exchange column. The water eluate was collected to give the decaffeinated theaflavin solution.

### Purification of Theaflavins and Thearubigins

The decaffeinated theaflavin solution was applied onto an adsorption resin (Model D201, Manufacturer: Xi'an Lanxiao Scientific Ltd, Xi'an, China). The resin was washed with water. The amount of water used was 5 times to the volume of resin. The tea compounds that were not assorted with resin were removed. Thearubigins of high molecular weight were eluted with 35% ethanol, which was the first elution liquid (1). Then, theaflavins and thearubigins of low molecular weight were eluted with 55% ethanol, which was the second elution liquid (2). The elution liquids were concentrated and alcohol recovered. The concentrated solutions were spray-dried and ground to give desired products.

From the first elution liquid (1), the content of high molecular weight thearubigins was above 70%. From the second elution liquid, the content of theaflavins was above 60% and low molecular weight thearubigins was above 30%.

### Example 3

### Treatment of the Starting Materials: Fresh Green Tea Leaves

1. Foreign matter and other impurities were removed from fresh tea leaves. Then, the fresh green tea leaves were washed and withered in a dry and weak light place for 12 h.
2. The withered green tea leaves were crushed.

### Tea Fermentation Process—Formation of Theaflavins and Thearubigins

1. The crushed leaves were put in a fermentation tank. Water was added in the tank in the amount of 8 times the volume of crushed tea leaves. Eggplant juice was added in the tank in the amount of 3.0 times the volume of crushed green tea leaves. At temperature 45° C., pH=5.5, air was introduced. This natural fermentation process was maintained for 40 min.
2. At the end of fermentation, the air flow was stopped and the temperature was raised to 100° C. rapidly and kept for 20 min. A solution of 95% ethanol was added to the tank and the entire mixture was stirred for 40 minutes. The ratio of 95% ethanol to the initial mixture was 3:1. Then, the mixture was centrifuged to separate the supernatant clear liquid and precipitates.
3. The precipitates were washed again in a tank with a solution of 50% ethanol for 30 min. The ratio of 50% ethanol to precipitates mixture was 3:1. This mixture was centrifuged to separate supernatant clear liquid from precipitates.
4. The supernatant liquids of step 2 and 3 were combined and then, concentrated to remove ethanol. The concentrate was centrifuged to separate supernatant clear liquid from precipitates. The precipitates were dried to yield a material containing more than 30% thearubigins.

### Decaffeination

The supernatant clear liquid obtained from step 4 was passed through a strong acid ion exchange column. The water eluate was collected to give the decaffeinated theaflavin solution.

### Purification of Theaflavins and Thearubigins

The decaffeinated theaflavin solution was applied onto an adsorption resin (Model 102, Manufacturer: Xi'an Lanxiao Scientific Ltd, Xi'an, China). The resin was washed with water. The amount of water used was 3 times to the volume of resin. The tea compounds that were not assorted with resin were removed. Thearubigins of high molecular weight were eluted with 25% ethanol, which was the first elution liquid (1). Then, theaflavins and thearubigins of low molecular weight were eluted with 50% ethanol, which was the second elution liquid (2). The elution liquids were concentrated and alcohol recovered. The concentrated solutions were spray-dried and grinded to give desired products.

From the first elution liquid (1), the content of high molecular weight thearubigins was above 70%. From the second elution liquid, the content of theaflavins was above 60% and low molecular weight thearubigins was above 30%.

1. A composition comprising at least 10% by weight of total theaflavins.

2. The composition of claim 1, wherein the composition is a black tea extract.

3. The composition of claim 1, wherein the composition is an oolong tea extract.

4. The composition of claim 1, wherein the composition is a green tea extract.

5. The composition of claim 1, wherein the composition is a nutraceutical.

6. The composition of claim 1, wherein the composition is an ethanol extract of tea.

7. The composition of claim 1, wherein the composition is free of ethyl acetate.

8. The composition of claim 1, wherein the composition is an extract of tea and comprises at least 1% by weight of theaflavin.

9. The composition of claim 1, wherein the composition is an extract of tea and comprises at least 5% by weight of theaflavin-3-gallate.

10. The composition of claim 1, wherein the composition is an extract of tea and comprises at least 1% by weight of theaflavin-3′-gallate.

11. The composition of claim 1, wherein the composition is an extract of tea and comprises at least 3% by weight of theaflavin-3,3′-digallate.

12. The composition of claim 1, wherein the composition is an extract of tea and comprises from about 12% to about 17% by weight theaflavin-3-gallate and from about 3% to about 8% theaflavin-3′-gallate.

13. The composition of claim 12, wherein the extract further comprises from about 9% to about 14% by weight theaflavin-3,3′-digallate.

14. The composition of claim 1, wherein the composition comprises at least 60% by weight of total theaflavins.

15. The composition of claim 1, further comprising a pharmaceutically acceptable carrier or diluent.

16. The composition of claim 15, wherein the carrier is a food product.

17. The composition of claim 15, wherein the carrier is a beverage.

18. The composition of claim 15, wherein the composition is formulated as a dietary supplement.

19. The composition of claim 15, wherein the composition is in the form of a capsule, a tablet, a lozenge or a coated tablet.

20. The composition of claim 15, wherein the composition is in the form of a solution, a syrup, or a suspension.

21. The composition of claim 1 wherein the composition comprises about 440 mg of the extract.

22. The composition of claim 1, wherein the composition comprises about 175 mg of total theaflavins.

23. The composition of claim 1, wherein the composition comprises less than 0.5% by weight caffeine.

24. A process of making a theaflavins enriched extract of tea having a low content of high molecular weight thearubigins which comprises extracting theaflavins from tea using ethanol to produce an extract having a high theaflavins content and a low content of high molecular weight thearubigins.

25-37. (canceled)
